SEQUENCE LISTING (16 sequences)

SEQ ID NO: 1 - DNA, 56 bp, Artificial Sequence
  Description of Artificial Sequence Target DNA
  a or g or t/u 54
  a or g or c or t/u, unknown or other 55
agagctacgagctgcctgacggccaggtcatcaccattggcaatgagcggttcdna

SEQ ID NO: 2 - DNA, 25 bp, Artificial Sequence
  Description of Artificial Sequence Donor probe a
gaaccgctcattgccaatggtgatg

SEQ ID NO: 3 - DNA, 25 bp, Artificial Sequence
  Description of Artificial Sequence Donor probe b
gtcaggcagctcgtagctcttctcc

SEQ ID NO: 4 - DNA, 8 bp, Artificial Sequence
  Description of Artificial Sequence Acceptor probe a
acctggcc

SEQ ID NO: 5 - DNA, 28 bp, Artificial Sequence
  Description of Artificial Sequence Acceptor probe b
acctggccgtcaggcagctcgtagctct

SEQ ID NO: 6 - DNA, 19 bp, Artificial Sequence
  Description of Artificial Sequence Target DNA-1
aactatacaacgggctgaa

SEQ ID NO: 7 - DNA, 27 bp, Artificial Sequence
  Description of Artificial Sequence Target DNA-2
aactatacaacgggctgaagggctgaa

SEQ ID NO: 8 - DNA, 35 bp, Artificial Sequence
  Description of Artificial Sequence Target DNA-3
aactatacaacgggctgaagggctgaagggctgaa

SEQ ID NO: 9 - DNA, 43 bp, Artificial Sequence
  Description of Artificial Sequence Target DNA-4
aactatacaacgggctgaagggctgaagggctgaagggctgaa

SEQ ID NO: 10 - DNA, 51 bp, Artificial Sequence
  Description of Artificial Sequence Target DNA-5
aactatacaacgggctgaagggctgaagggctgaagggctgaagggctgaa

SEQ ID NO: 11 - DNA, 8 bp, Artificial Sequence
  Description of Artificial Sequence Donor probe
ttcagccc

SEQ ID NO: 12 - DNA, 11 bp, Artificial Sequence
  Description of Artificial Sequence Acceptor probe
gttgtatagtt

SEQ ID NO: 13 - DNA, 22 bp, Artificial Sequence
  Description of Artificial Sequence Target DNA-1
tgaggtagtaggttgtatagtt

SEQ ID NO: 14 - DNA, 11 bp, Artificial Sequence
  Description of Artificial Sequence Donor probe-1
ctactacctca

SEQ ID NO: 15 - DNA, 11 bp, Artificial Sequence
  Description of Artificial Sequence Acceptor probe-1
aactatacaac

SEQ ID NO: 16 - DNA, 22 bp, Artificial Sequence
  Description of Artificial Sequence Target DNA-2
gttgtatagtttgaggtagtag

(19) United States
(12) Patent Application Publication
(10) Pub. No.: US 2019/0271027 A1
(43) Pub. Date: Sep. 5, 2019

(54) METHOD FOR DETECTING TARGET NUCLEIC ACID MOLECULE

(71) Applicants: OLYMPUS CORPORATION, Tokyo (JP); RIKEN, Wako-shi (JP)

(72) Inventors: Takuya Hanashi, Tokyo (JP); Tetsuya Tanabe, Tokyo (JP); Takeshi Hanami, Yokohama-shi (JP); Yoshihide Hayashizaki, Tokyo (JP)

(73) Assignees: OLYMPUS CORPORATION, Tokyo (JP); RIKEN, Wako-shi (JP)

(21) Appl. No.: 16/382,532

(22) Filed: Apr. 12, 2019

Related U.S. Application Data
(63) Continuation of application No. PCT/JP2016/080743, filed on Oct. 17, 2016.

(51) Int. Cl. C12Q 1/6818; C12Q 1/6876; G01N 21/64

(52) U.S. Cl. CPC C12Q 1/6818; C12Q 1/6876; G01N 2021/6441; C12Q 2600/158; C12Q 2563/107; G01N 21/6428

(57) ABSTRACT

A method for detecting a target nucleic acid molecule of the present invention includes a step of associating a first and third probes labeled with a first fluorescent substance which is an energy donor with a second probe labeled with a second fluorescent substance which is an energy acceptor to form an associate in a nucleic acid molecule; and a step of emitting light with an excitation wavelength of the first fluorescent substance to the associate to detect the target nucleic acid molecule using fluorescence released from the second fluorescent substance in the associate as an indicator, wherein a region associating with the second probe is between a region associating with the first probe and a region associating with the third probe in the target nucleic acid molecule.

### BACKGROUND OF THE INVENTION

### Field of the Invention

The present invention relates to a method for detecting a target nucleic acid molecule with high sensitivity by using fluorescence resonance energy transfer (FRET).

### Description of Related Art

As a method for detecting a nucleic acid having a specific base sequence, methods have been frequently reported for examining a base sequence of a nucleic acid by using artificially synthesized short-chain oligonucleotides such as probes and primers. In particular, in genetic analysis such as somatic cell mutation and single nucleotide polymorphism, various methods using fluorescence have been developed because of excellent detection sensitivity.

For example, a method for detecting a target nucleic acid molecule by utilizing FRET is known (for example, refer to PTL 1). In this method, a donor probe to which a fluorescent substance (donor pigment), which is an energy donor for FRET, is bound; and an acceptor probe to which a fluorescent substance (acceptor pigment), which is an energy acceptor, is bound, associate (hybridize) with two adjacent regions of the target nucleic acid molecule, respectively. In an associate obtained by both the donor probe and the acceptor probe associating with the target nucleic acid molecule, since the acceptor pigment and the donor pigment are close to each other, in a case where the associate is irradiated with light with an excitation wavelength of the donor pigment, FRET occurs from the excited donor pigment, and thereby generating fluorescence from the acceptor pigment. Meanwhile, FRET does not occur in the target nucleic acid molecule to which any one of the probes is not bound, and therefore fluorescence from the acceptor pigment cannot be obtained. As described above, since fluorescence from the acceptor pigment can be observed only in the case where the target nucleic acid molecule, which is a detection target, is present, this method is used for detection and quantification of the target nucleic acid molecule.

In addition, as a method for detecting a target nucleic acid molecule with only one kind of probe without utilizing FRET, PTLs 2 and 3 disclose a method in which a nucleic acid probe labeled with a fluorescent atomic group exhibiting an excitonic effect (exciton coupling) is used. The excitonic effect is, for example, an effect in which a plurality of pigments aggregate in parallel to form an H-aggregate, and therefore almost no fluorescence emission is shown. A probe (E probe) having an excitonic effect releases almost no fluorescence in a free state, but by the probe associating with a target nucleic acid molecule, the H-aggregate dissociates, thereby emitting fluorescence.

[PTL 1] Japanese Patent No. 4118932

[PTL 2] Japanese Unexamined Patent Application, First Publication No. 2013-183736

[PTL 3] PCT International Publication No. WO2014/034818

### SUMMARY OF THE INVENTION

In a case where a nucleic acid molecule is detected using a FRET probe, light intensity of fluorescence to be detected becomes significantly smaller than that in a case of detection using a probe labeled with a single fluorescent pigment used in general, and therefore detection efficiency of the target nucleic acid molecule decreases.

An object of the present invention is to provide a method for detecting a target nucleic acid molecule with high sensitivity by utilizing FRET, and a probe set used in the method.

As a result of extensive research to solve the above-mentioned problems, the inventors of the present invention have found the following and therefore have completed the present invention. An associate is formed such that two donor probes are disposed at a position of sandwiching one acceptor probe therebetween in a target nucleic acid molecule, and fluorescence resonance energy is supplied from the two donor probes to the one acceptor probe, thereby intensifying the luminance of the fluorescence from the acceptor probe.

That is, a method for detecting a target nucleic acid molecule and a probe set according to the present invention are the following [1] to [11].

[1] A method for detecting a target nucleic acid molecule, including:

a step (a) of mixing, into a nucleic acid-containing sample, a first probe labeled with a first fluorescent substance which is an energy donor in a fluorescence resonance energy transfer phenomenon, a second probe labeled with a second fluorescent substance which is an energy acceptor in the fluorescence resonance energy transfer phenomenon, and a third probe labeled with the first fluorescent substance so as to prepare a sample solution;

a step (b) of allowing the target nucleic acid molecule in the sample solution prepared in the step (a) to associate with the first probe, the second probe, and the third probe so as to form an associate made of the first probe, the second probe, the third probe, and the target nucleic acid molecule; and

a step (c) of emitting light with an excitation wavelength of the first fluorescent substance to the sample solution after the step (b) so as to detect the target nucleic acid molecule using fluorescence released from the second fluorescent substance in the associate as an indicator,

in which a region associating with the second probe is between a region associating with the first probe and a region associating with the third probe in the target nucleic acid molecule.

[2] The method for detecting a target nucleic acid molecule according to [1], in which the first probe, the second probe, or the third probe is a probe in which emission luminance changes according to a state of associating or not associating with the target nucleic acid molecule.

[3] The method for detecting a target nucleic acid molecule according to [2], in which the first fluorescent substance or the second fluorescent substance is a fluorescent atomic group exhibiting excitonic effects.

[4] The method for detecting a target nucleic acid molecule according to any one of [1] to [3],

in which a distance between a base in the target nucleic acid molecule with which a base to which the first fluorescent substance in the first probe is bound is associated, and a base in the target nucleic acid molecule with which a base to which the second fluorescent substance in the second probe is bound is associated, is 8 bases or less, and

a distance between a base in the target nucleic acid molecule with which a base to which the first fluorescent substance in the third probe is bound is associated, and the base in the target nucleic acid molecule with which the base to which the second fluorescent substance in the second probe is bound is associated, is 8 bases or less.

[5] The method for detecting a target nucleic acid molecule according to any one of [1] to [4], in which a base length of the second probe is 5 to 17 bases.

[6] The method for detecting a target nucleic acid molecule according to any one of [1] to [5],

in which a first target nucleic acid molecule that associates with the first probe, the second probe, and the third probe; and a second target nucleic acid molecule that binds to only one of the first probe and the third probe, and to the second probe are contained in the nucleic acid-containing sample,

in the step (b), a first associate obtained by associating the first target nucleic acid molecule with the first probe, the second probe, and the third probe; and a second associate obtained by associating the second target nucleic acid molecule with the second probe, and any one of the first probe and the third probe are formed, and

in the step (c), the light with the excitation wavelength of the first fluorescent substance is emitted to the sample solution, and the first target nucleic acid molecule and the second target nucleic acid molecule are distinctively detected using fluorescence luminance released from the second fluorescent substance in the associate of one molecule as an indicator so as to calculate an abundance ratio of the first target nucleic acid molecule and the second target nucleic acid molecule in the sample solution.

[7] A probe set for detecting a target nucleic acid molecule, including:

a first probe in which a single-stranded nucleic acid molecule associating with the target nucleic acid molecule is labeled with a first fluorescent substance which is an energy donor in a fluorescence resonance energy transfer phenomenon;

a second probe in which a single-stranded nucleic acid molecule associating with the target nucleic acid molecule is labeled with a second fluorescent substance which is an energy acceptor in the fluorescence resonance energy transfer phenomenon; and

a third probe in which a single-stranded nucleic acid molecule associating with the target nucleic acid molecule is labeled with the first fluorescent substance,

in which the first probe, the second probe, and the third probe are capable of forming, with the target nucleic acid molecule, an associate in which the third probe is disposed on a side opposite to the first probe based on the second probe, and

in a case where light with an excitation wavelength of the first fluorescent substance is emitted to the associate, a fluorescence resonance energy transfer occurs between the first fluorescent substance in the first probe and the second fluorescent substance in the second probe, and between the first fluorescent substance in the third probe and the second fluorescent substance in the second probe, and fluorescence released from the second fluorescent substance is detected.

[8] The probe set according to [7], in which the first probe, the second probe, or the third probe is a probe in which emission luminance changes according to a state of associating or not associating with the target nucleic acid molecule.

[9] The probe set according to [8], in which the first fluorescent substance or the second fluorescent substance is a fluorescent atomic group exhibiting excitonic effects.

[10] The probe set according to any one of [7] to [9],

in which, in the associate,

a distance between a base in the target nucleic acid molecule with which a base to which the first fluorescent substance in the first probe is bound is associated, and a base in the target nucleic acid molecule with which a base to which the second fluorescent substance in the second probe is bound is associated, is 8 bases or less, and

a distance between a base in the target nucleic acid molecule with which a base to which the first fluorescent substance in the third probe is bound is associated, and the base in the target nucleic acid molecule with which the base to which the second fluorescent substance in the second probe is bound is associated, is 8 bases or less.

[11] The probe set according to any one of [7] to [10], in which a base length of the second probe is 5 to 17 bases.

In the method for detecting a target nucleic acid molecule according to the present invention, because fluorescence resonance energy is supplied from two donor probes with respect to one acceptor probe, fluorescence luminance emitted from an associate formed of the target nucleic acid molecule, the acceptor probe, and the donor probe is large compared to a detection method utilizing FRET of the related art which uses one acceptor probe and one donor probe. Accordingly, by using the detection method, detection efficiency of the target nucleic acid molecule can be enhanced.

In addition, by using the probe set according to the present invention, the detection method can be simply and easily carried out.

### BRIEF DESCRIPTION OF DRAWINGS

FIG. 1 is a schematic view of an associate formed by a target nucleic acid molecule and each probe of a detection method using a FRET probe of the related art (left) and of a detection method according to the present invention (right).

FIG. 2 is a graph showing measurement results of fluorescence luminance of each sample solution in Example 1.

FIG. 3 is a graph showing measurement results of the number of molecules of associates in each sample solution measured by SSMC in Example 1.

FIG. 4 is a graph showing measurement results of fluorescence luminance of each sample solution in Reference Example 1.

FIG. 5 is a graph showing measurement results of the number of molecules of associates in each sample solution measured by SSMC in Reference Example 1.

FIG. 6 is a graph showing measurement results of the number of molecules of associates in each sample solution measured by SSMC in Reference Example 2.

### DETAILED DESCRIPTION OF THE INVENTION

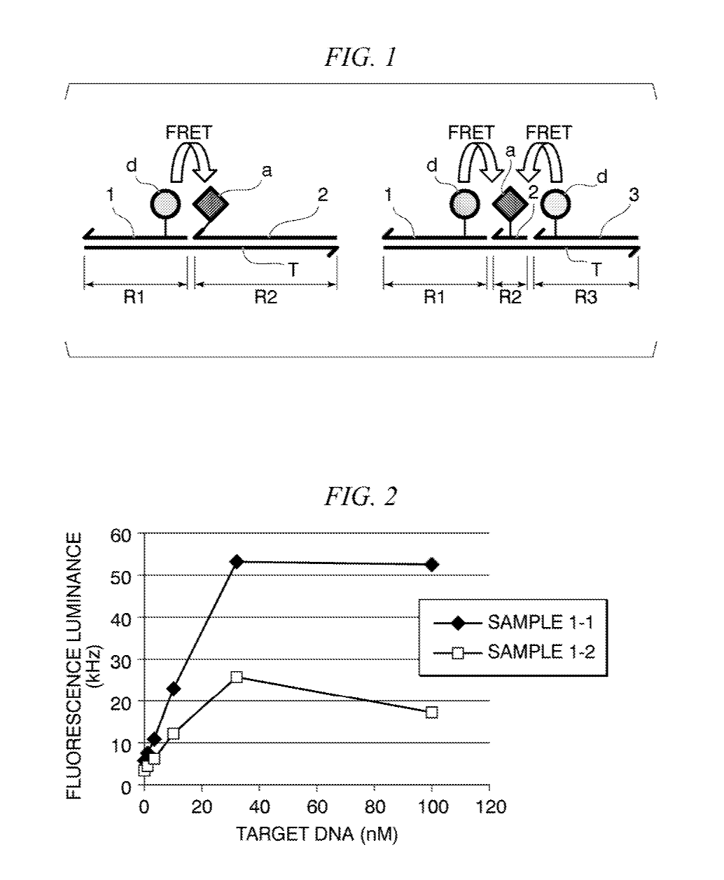

A method for detecting a target nucleic acid molecule according to the present invention (hereinafter referred to as “detection method according to the present invention”) is a method for detecting a target nucleic acid molecule using a FRET probe and is characterized by using a first probe labeled with a first fluorescent substance which is an energy donor in a FRET phenomenon, a second probe labeled with a second fluorescent substance which is an energy acceptor in the FRET phenomenon, and a third probe labeled with the first fluorescent substance. Hereinafter, “first fluorescent substance” may be referred to as “donor fluorescent substance” and “second fluorescent substance” may be referred to as “acceptor fluorescent substance” in some cases. The first probe and the third probe labeled with the donor fluorescent substance are so-called donor probes, and the second probe labeled with the acceptor fluorescent substance is a so-called acceptor probe. In the detection method according to the present invention, two donor probes are disposed so as to sandwich the acceptor probe therebetween, and the one acceptor probe and the two donor probes associate with one molecule of the target nucleic acid molecule. FIG. 1 is a schematic view of an associate formed by a target nucleic acid molecule and each probe of a detection method using a FRET probe of the related art and of the detection method according to the present invention. In the method of the related art, with respect to one molecule of the target nucleic acid molecule, one acceptor probe and one donor probe are associated so as to be adjacent to each other, thereby causing FRET (left drawing). In contrast, in the method for detecting a target nucleic acid molecule according to the present invention, with respect to the target nucleic acid molecule, two donor probes (the first probe and the third probe) are disposed so as to sandwich the acceptor probe (the second probe), and therefore fluorescence resonance energy is supplied to one acceptor probe from two donor probes (right drawing). Theoretically, an amount of fluorescence resonance energy supplied to the acceptor is about twice that in the method of the related art, and therefore luminance of fluorescence released from the acceptor is intensified, thereby improving detection sensitivity of the associate.

The donor fluorescent substance and the acceptor fluorescent substance used in the present invention can be appropriately selected from substances generally used in the FRET probe as long as they are any combination of substances which cause FRET in a case where the substances are sufficiently close to each other, and are substances not inhibiting the formation of the associate. For example, it is possible to use a combination of PE (phycoerythrin) as the donor fluorescent substance, and Cy5, Cy5.5, Texas Red (registered trademark), Alexa fluor (registered trademark) 610, Alexa fluor 647, and Alexa fluor 680 as the acceptor fluorescent substance. In addition, it is possible to use a combination of APC (allophycocyanin) as the donor fluorescent substance and Cy5.5 as the acceptor fluorescent substance.

As the first probe, the second probe, and the third probe used in the present invention, probes in which emission luminance changes according to a state of associating or not associating with the target nucleic acid molecule can be used. For example, by using a probe in which emission intensity is small in the state of not associating with the target nucleic acid molecule, and emission intensity is large in the state of associating with the target nucleic acid molecule, it is possible to suppress background and noise when detecting fluorescence of FRET, and to further enhance the detection sensitivity of the target nucleic acid molecule.

For example, by using a fluorescent atomic group exhibiting excitonic effects as the donor fluorescent substance or the acceptor fluorescent substance, the probe can be the probe in which emission luminance changes according to the state of associating or not associating with the target nucleic acid molecule. Both the donor fluorescent substance and the acceptor fluorescent substance may be fluorescent atomic groups exhibiting excitonic effects, and any one thereof may be a fluorescent atomic group exhibiting excitonic effects. In a case where any one thereof is the fluorescent atomic group, it is preferable that the donor fluorescent substance be the fluorescent atomic group exhibiting excitonic effects, because the background and noise can be suppressed more efficiently.

Examples of the fluorescent atomic group exhibiting excitonic effects include thiazole orange and derivatives thereof, oxazole yellow and derivatives thereof, cyanine and derivatives thereof, hemicyanine and derivatives thereof, methyl red and derivatives thereof, and pigment groups generally called cyanine pigments and azo pigments. Examples of the cyanine pigments include Cy5, Cy5.5, and the like. In addition, a pigment known as a fluorescent pigment which changes fluorescence intensity by binding to a nucleic acid such as DNA, a fluorescent pigment in which fluorescence intensity changes according to microscopic polarity, and a group derived therefrom can also be appropriately used. Examples of the fluorescent pigment that changes the fluorescence intensity by binding to a nucleic acid include ethidium bromide. Examples of the fluorescent pigment in which the fluorescence intensity changes according to microscopic polarity include pyrenecarboxamide and prodane. In addition, fluorescein can also be used as the fluorescent atomic group exhibiting excitonic effects.

The probe exhibiting excitonic effects may bind a fluorescent atomic group exhibiting one excitonic effect to a single-stranded nucleic acid molecule for association with the target nucleic acid molecule, directly or indirectly via a suitable linker, or may bind a fluorescent atomic group exhibiting at least two excitonic effects to the single-stranded nucleic acid molecule for association with the target nucleic acid molecule, in a state of being close to each other. Examples of the probe that binds the fluorescent atomic group exhibiting two excitonic effects to the single-stranded nucleic acid molecule, in a state of being close to each other, include a probe in which a group composed of a structure represented by General Formula (1) is bonded to one base in the single-stranded nucleic acid molecule for association with the target nucleic acid molecule (refer to PTL 2).

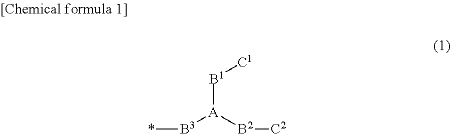

In General Formula (1), A is CR, N, P, PO, B, or SiR, and R is a hydrogen atom, an alkyl group or any substituent.

In General Formula (1), B¹, B², and B³ are linkers (bridging atoms or atomic groups), and a main chain length is arbitrary. The main chain may or may not contain C, N, O, P, B. Si, or S, and may or may not contain each of a single bond, a double bond, a triple bond, an amide bond, an ester bond, a disulfide bond, an imino group, an ether bond, a thioether bond, and a thioester bond. B¹, B², and B³ may be the same or different from each other. A group composed of the structure of General Formula (1) is bonded to a side chain in a base constituting the s single-stranded nucleic acid molecule in B³ (asterisk in Formula (1)).

In General Formula (1), C¹ and C² are the fluorescent atomic groups exhibiting excitonic effects, and may be the same or different from each other. Examples of the fluorescent atomic groups exhibiting excitonic effects include those listed above. In addition, examples thereof can include an atomic group represented by General Formula (2) or General Formula (3).

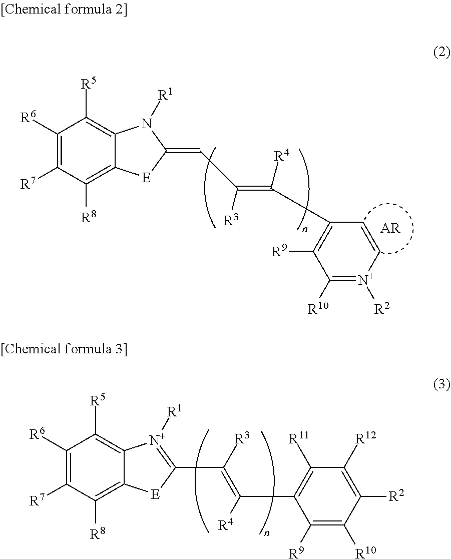

In General Formulas (2) and (3). E is O, S, and Se, and n is 0 or a positive integer. One of R¹ and R² is a linking group bonded to B¹ or B² in General Formula (1), and the other is a hydrogen atom or a lower alkyl group. R³ to R¹² each independently represents a hydrogen atom, a halogen atom, an alkyl group, an alkoxy group, a nitro group, a cyano group, a carbonyl group, a carboxyl group, an amino group, a silyl group, or a boryl group. AR is an arbitrary aromatic ring, which may or may not be present. In a case where a plurality of R³s are present in General Formula (2) or (3), the plurality of R³s may be the same or different from each other. In a case where a plurality of R⁴s are present in General Formula (2) or (3), the plurality of R⁴s may be the same or different from each other.

In a case where C¹ or C² in General Formula (1) is the atomic group represented by General Formula (2) or General Formula (3), E, AR, n, and R¹ to R² in C^{t}; and E, AR, n, and R¹ to R² in C² may be the same as or different from each other.

The first probe and the third probe are each obtained by labeling the single-stranded nucleic acid molecule associating with the target nucleic acid molecule with the donor fluorescent substance. Similarly, the second probe is obtained by labeling the single-stranded nucleic acid molecule associating with the target nucleic acid molecule with the acceptor fluorescent substance. Each probe may bind the donor fluorescent substance or the acceptor fluorescent substance to the single-stranded nucleic acid molecule associating with the target nucleic acid molecule, directly or indirectly via a linker. Examples of the linker include a single-stranded nucleic acid molecule with 1 to 10 bases in length. The binding of the donor fluorescent substance or the acceptor fluorescent substance to the single-stranded nucleic acid molecule associating with the target nucleic acid molecule or to the linker can be carried out by a general method.

Abase sequence of the single-stranded nucleic acid molecule associating with the target nucleic acid molecule in each probe is designed based on base sequence information of the target nucleic acid molecule. In designing, it is also possible to use generally used primer-probe design software and the like.

The first probe, the second probe, and the third probe form, with the target nucleic acid molecule, the associate in which the third probe is disposed on a side opposite to the first probe based on the second probe. For this reason, a base sequence of the single-stranded nucleic acid molecule associating with the target nucleic acid molecule of each probe is designed such that a region (“R2” in FIG. 1) associating with the second probe is set between a region associating with the first probe (“R1” in FIG. 1) and a region associating with the third probe (“R3” in FIG. 1) in the target nucleic acid molecule. In the target nucleic acid molecule, the region associating with the second probe and the region associating with the first probe may be adjacent to each other, or may be separated by 1 to 5 bases. Similarly, in the target nucleic acid molecule, the region associating with the second probe and the region associating with the third probe may be adjacent to each other, or may be separated by 1 to 5 bases.

In the association between the target nucleic acid molecule and the three probes, FRET is required to occur between the donor fluorescent substance in the first probe and the acceptor fluorescent substance in the second probe, and between the donor fluorescent substance in the third probe and the acceptor fluorescent substance in the second probe. Accordingly, the region associating with the first probe, the region associating with the second probe, and the region associating with the third probe in the target nucleic acid molecule are designed such that a distance between the donor fluorescent substance in the first probe and the acceptor fluorescent substance in the second probe is sufficiently short for FRET to occur, and a distance between the donor fluorescent substance in the third probe and the acceptor fluorescent substance in the second probe is sufficiently short for FRET to occur. For example, the region associating with the first probe and the region associating with the second probe in the target nucleic acid molecule are designed such that a distance between a base in the target nucleic acid molecule with which a base to which the donor fluorescent substance in the first probe is bound is associated, and a base in the target nucleic acid molecule with which the base to which the acceptor fluorescent substance in the second probe is bound is associated, is 8 bases or less, preferably 6 bases or less, and more preferably 4 bases or less. For example, the region associating with the third probe and the region associating with the second probe in the target nucleic acid molecule are designed such that a distance between a base in the target nucleic acid molecule with which a base to which the donor fluorescent substance in the third probe is bound is associated, and the base in the target nucleic acid molecule with which the base to which the acceptor fluorescent substance in the second probe is bound is associated, is 8 bases or less, preferably 6 bases or less, and more preferably 4 bases or less. Therefore, it is preferable that the base to which the acceptor fluorescent substance in the second probe is bound be at or near the center of the probe, and a base length be preferably 5 to 17 bases.

The base sequence of the single-stranded nucleic acid molecule associating with the target nucleic acid molecule in each probe may be any sequence that can specifically associate with an objective region of the target nucleic acid molecule. For example, the base sequence of the single-stranded nucleic acid molecule associating with the target nucleic acid molecule in the first probe is preferably a base sequence complementary to the region associating with the first probe designed in the target nucleic acid molecule, or may be a base sequence having mismatches in which 1 to 5 bases are substituted, deleted, or inserted with respect to a sequence complementary to the base sequence of the corresponding region. Similarly, each of base sequences of the single-stranded nucleic acid molecule associating with the target nucleic acid molecule in the second probe and the third probe is preferably a base sequence complementary to the region associating with the second probe and the region associating with the third probe designed in the target nucleic acid molecule, or may be a base sequence having mismatches in which 1 to bases are substituted, deleted, or inserted with respect to a sequence complementary to the base sequence of the corresponding region.

A base length of the single-stranded nucleic acid molecule associating with the target nucleic acid molecule in the first probe and the third probe is not particularly limited as long as it is a length that can specifically associate with an objective region of the target nucleic acid molecule, and this also applies to general probes. Specifically, for example, a base length can be 15 to 50 bases and is preferably 15 to 35 bases.

The target nucleic acid molecule may be composed only of naturally occurring nucleotides such as DNA, RNA, and 2′-O-methyl RNA, or may be an artificial nucleic acid molecule in which a partial or entire part thereof contains artificial nucleotides.

The single-stranded nucleic acid molecule associating with the target nucleic acid molecule in each probe may be composed only of naturally occurring nucleotides such as DNA, RNA, and 2′-O-methyl RNA, or may be an artificial nucleic acid molecule in which a partial or entire part thereof contains artificial nucleotides.

The term “artificial nucleotide” means that a nucleotide is an artificially synthesized nucleotide, which has a structure different from that of the naturally occurring nucleotide but which can function similarly to the naturally occurring nucleotide. The phrase “can function similarly to the naturally occurring nucleotide” means that a nucleic acid molecule can be formed by a phosphodiester bond or the like similarly to the naturally occurring nucleotide.

Examples of such an artificial nucleotide include Bridged nucleic acid (BNA), 2′-O,4′-C-ethylene-bridged nucleic acid (ENA), peptide nucleic acid (PNA), glycol nucleic acid (GNA), threose nucleic acid (TNA), Hexitol Nucleic Acid (HNA), and the like. BNA is a nucleic acid in which a part of a naturally occurring nucleotide is crosslinked and examples thereof include Locked nucleic acid (LNA) which is a bridged artificial nucleotide in which an oxygen atom at the 2′ position and a carbon atom at the 4′ position of a ribose ring are bonded via methylene.

In particular, because a base length of the single-stranded nucleic acid molecule associating with the target nucleic acid molecule in the second probe is relatively short, in order to specifically associate with an objective region of the target nucleic acid molecule, it is preferable to include an artificial nucleotide in which artificial recognition ability is improved, such as LNA and ENA.

The detection method according to the present invention has the following steps (a) to (c).

The step (a) of mixing the first probe, the second probe, and the third probe into a nucleic acid-containing sample so as to prepare a sample solution.

The step (b) of allowing the target nucleic acid molecule in the sample solution prepared in the step (a) to associate with the first probe, the second probe, and the third probe so as to form an associate made of the first probe, the second probe, the third probe, and the target nucleic acid molecule.

The step (c) of emitting light with an excitation wavelength of the first fluorescent substance to the sample solution after the step (b) so as to detect the target nucleic acid molecule using fluorescence released from the second fluorescent substance in the associate as an indicator.

In the present invention, the target nucleic acid molecule means a nucleic acid molecule having a specific base sequence (target base sequence) which is a target of detection. The target nucleic acid molecule is not particularly limited as long as base sequence information is clear to the extent that the first probe and the like can be designed. For example, the target nucleic acid molecule may be a nucleic acid molecule having a base sequence present in a chromosome of an animal or a plant or in a gene of a bacterium or a virus; or may be a nucleic acid molecule having an artificially designed base sequence. In the present invention, the target nucleic acid molecule may be a double-stranded nucleic acid or a single-stranded nucleic acid. In addition, the target nucleic acid molecule may be any of DNA or RNA. Examples of the target nucleic acid molecule include mRNA, hnRNA, genomic DNA, synthetic DNA by PCR amplification and the like, cDNA synthesized from RNA using reverse transcriptase, and the like.

In addition, in the present invention, the nucleic acid-containing sample is not particularly limited as long as it is a sample containing a nucleic acid molecule. Examples of the nucleic acid-containing sample include a biological sample collected from an animal or the like, a sample prepared from cultured cells or the like, a reaction solution after a nucleic acid synthesis reaction, and the like. The nucleic acid-containing sample may be a biological sample or the like itself, or may be a nucleic acid solution extracted and purified from a biological sample or the like.

First, as the step (a), the sample solution is prepared by mixing the three probes into the nucleic acid-containing sample. In this case, an appropriate solvent may be added as necessary. The solvent is not particularly limited as long as it is a solvent that does not inhibit FRET between the donor fluorescent substance and the acceptor fluorescent substance, and the detection of fluorescence emitted from the acceptor fluorescent substance, and can be selected appropriately from among buffers generally used in the present field. Examples of the buffer include a phosphate buffer such as phosphate-buffered saline ((PBS), pH 7.4), a tris buffer, and the like.

In addition, in order to suppress non-specific association between each probe and a nucleic acid molecule other than the target nucleic acid molecule, it is preferable to add a surfactant, formamide, dimethylsulfoxide, urea, or the like into the sample solution in advance. Only one kind of these compounds may be added, or two or more kinds thereof may be added in combination. By adding these compounds, it is possible to make non-specific association less likely to occur in a relatively low-temperature environment.

Next, as the step (b), the target nucleic acid molecule in the sample solution prepared in the step (a) associates with the three probes to form an associate. In order that the target nucleic acid molecule specifically associates with each probe, firstly, all nucleic acid molecules in the sample solution are denatured, and then an associate is formed.

In the present invention, the phrase “to denature nucleic acid molecules” means that base pairs are dissociated. In the present invention, because the influence on the donor fluorescent substance and the acceptor fluorescent substance is relatively small, it is preferable to perform denaturation (heat denaturation) by high-temperature treatment or denaturation by low-salt-concentration treatment. Among them, it is preferable to perform heat denaturation because the operation is simple. Denaturing conditions depend on the target nucleic acid molecule and the base sequence and base length of each probe. For example, in regard to the heat denaturation, in general, the nucleic acid molecules can be denatured by incubating the sample solution at a temperature of 90° C. to 100° C. in a case where the target nucleic acid molecule or the probe is DNA and at 70° C. in a case of RNA, for several seconds to about 2 minutes. Meanwhile, denaturation by low-salt-concentration treatment can be carried out by, for example, diluting with purified water or the like so that a salt concentration of the sample solution adjusted to become sufficiently low.

In the case of performing the heat denaturation, after the high-temperature treatment, by lowering a temperature of the sample solution to a temperature at which the target nucleic acid molecule and each probe can associate with each other, it is possible to form an associate composed of the target nucleic acid molecule, the first probe, the second probe, and the third probe in the sample solution. Specifically, a temperature is lowered to a temperature of ±3° C. of a Tm value (temperature at which 50% of double-stranded DNA dissociates into single-stranded DNA) of the single-stranded nucleic acid molecule associating with the target nucleic acid molecule in each probe. In a case where a Tm value of the single-stranded nucleic acid molecule associating with the target nucleic acid molecule is different for each probe, a temperature is lowered to a temperature of the lowest temperature Tm value ±3° C. Meanwhile, also in a case where denaturation by low-salt-concentration treatment is carried out, similarly, by adding a salt solution, or the like after the low-salt-concentration treatment, by increasing the salt concentration of the sample solution to a concentration at which the target nucleic acid molecule and each probe can associate with each other, it is possible to form an associate composed of the target nucleic acid molecule, the first probe, the second probe, and the third probe in the sample solution. A Tm value of the single-stranded nucleic acid molecule associating with the target nucleic acid molecule of each probe can be calculated by using a generally used primer-probe designing software or the like.

After the step (b), as the step (c), light with an excitation wavelength of the donor fluorescent substance is emitted to the sample solution so as to detect fluorescence of a fluorescence wavelength of the acceptor fluorescent substance. When the associate composed of the target nucleic acid molecule, the first probe, the second probe, and the third probe in the sample solution is irradiated with the light with the excitation wavelength of the donor fluorescent substance, FRET occurs between the donor fluorescent substance in the first probe and the acceptor fluorescent substance in the second probe and between the donor fluorescent substance in the third probe and the acceptor fluorescent substance in the second probe, and therefore, in the associate, fluorescence is released from the acceptor fluorescent substance. In other words, by measuring a fluorescence signal of the fluorescence wavelength of the acceptor fluorescent substance detected by release of the light with the excitation wavelength of the donor fluorescent substance, the associate composed of the target nucleic acid molecule, the first probe, the second probe, and the third probe can be detected. In a case where the target nucleic acid molecule is contained in the nucleic acid-containing sample, and in a case where the light with the excitation wavelength of the donor fluorescent substance is emitted, fluorescence of the fluorescence wavelength of the acceptor fluorescent substance is detected. Meanwhile, in a case where the target nucleic acid molecule is not contained in the nucleic acid-containing sample, and in a case where the light with the excitation wavelength of the donor fluorescent substance is emitted, fluorescence of the fluorescence wavelength of the acceptor fluorescent substance is not detected.

A method of measuring a fluorescence signal of a fluorescence wavelength of the acceptor fluorescent substance released from the associate containing the target nucleic acid molecule in the sample solution is not particularly limited, and may be a method for measuring fluorescence intensity of the entire sample solution, or may be a method in which molecules emitting fluorescence in the sample solution are detected and measured for each molecule.

The fluorescence intensity of the sample solution can be measured by a general method using a fluorescence spectrophotometer such as a fluorescence plate reader, or the like. The fluorescence intensity of the fluorescent wavelength of the acceptor fluorescent substance of the sample solution depends on an amount of the associate composed of the target nucleic acid molecule and the three probes contained in the sample solution. For this reason, for example, by creating a calibration curve showing a relationship between an amount of the acceptor fluorescent substance to be detected and fluorescence intensity in advance, an amount of the associate containing the target nucleic acid molecule in the sample solution, that is, an amount of the target nucleic acid molecule in the nucleic acid-containing sample can be quantified.

Examples of the method for measuring a fluorescence signal for each molecule in the sample solution include Scanning single-molecule counting (SSMC) (WO2012/102260), Fluorescence Correlation Spectroscopy (FCS), Fluorescence Intensity Distribution Analysis (FIDA), and FIDA polarization (FIDA-PO). Among them, because the detection efficiency of the target nucleic acid molecule can be markedly enhanced as compared with the method of the related art which uses one donor probe, measurement with SSMC is preferable.

Such detection and analysis of a fluorescence signal of one molecule can be carried out by a general method using a known single-molecule fluorescence spectroscopy system such as MF20 (manufactured by Olympus Corporation), or the like.

In addition, in the associate formed by the two donor probes (first probe and third probe), one acceptor probe (second probe), and the target nucleic acid molecule, luminance of fluorescence released from the acceptor by FRET is stronger than that of an associate formed by one donor probe (first probe or third probe), one acceptor probe (second probe), and the target nucleic acid molecule. In the detection method according to the present invention, two types of target nucleic acid molecules (first target nucleic acid molecule and second target nucleic acid molecule) having similar base sequences can be distinctively detected by utilizing a difference in the intensity of luminance.

Specifically, firstly, designing is performed such that, in the first target nucleic acid molecule, the associate can be formed by association with all of three probes, which are the first probe, the second probe, and the third probe, while the second target nucleic acid molecule associates with the second probe, and associates with only one of the first probe and the third probe. In a case where the first target nucleic acid molecule and the second target nucleic acid molecule are contained in the nucleic acid-containing sample subjected to the step (a), a first associate obtained by associating the first target nucleic acid molecule with the first probe, the second probe, and the third probe; and a second associate obtained by associating the second target nucleic acid molecule with the second probe, and with any one of the first probe and the third probe are formed, respectively in the step (b), in the sample solution prepared in the step (a).

Subsequently, in the step (c), in a case where the light with the excitation wavelength of the first fluorescent substance is emitted to the sample solution, luminance of the fluorescence released from the acceptor in the associate is greater in the first associate containing two donor probes in the associate compared with the second associate containing only one donor probe in the associate. Using the luminance of the fluorescence released from the acceptor (second fluorescent substance) in the associate of one molecule as an indicator, the first target nucleic acid molecule contained in the first associate and the second target nucleic acid molecule contained in the second associate can be distinctively detected. In a case where a fluorescence signal is measured for each molecule in the sample solution, a molecule with brighter luminance of fluorescence released from the acceptor by FRET is the first associate containing the first target nucleic acid molecule, and a darker molecule is the second associate containing the second target nucleic acid molecule. In a case where the first associate and the second associate are distinctively detected using a luminance value of the fluorescence released from the acceptor as the indicator, an abundance ratio of the first target nucleic acid molecule and the second target nucleic acid molecule in the sample solution can be calculated from the number of molecules of the first target nucleic acid molecule and the number of molecules of the second target nucleic acid molecule in the sample solution.

The detection method according to the present invention can also be used for detection of gene polymorphism. For example, among gene polymorphisms, using a mutant type as the first target nucleic acid molecule, and a wild type as the second target nucleic acid molecule, the first probe and the second probe are designed to associate with a region in which a base sequence is common in the mutant type and the wild type, and the third probe is designed to associate with the first target nucleic acid molecule which is the mutant type but not to associate with the second target nucleic acid molecule which is the wild type. Accordingly, an associate containing the wild-type target nucleic acid molecule is detected as a molecule with weak fluorescence luminance, and an associate containing the mutant-type target nucleic acid molecule is detected as a molecule with bright fluorescence luminance. In addition, based on the detection results obtained, an abundance ratio between the mutant-type nucleic acid molecule and the wild-type nucleic acid molecule in the nucleic acid-containing sample can be obtained.

It is also preferable to set the first probe, the second probe, and the third probe as a set. Using the probe set including these three probes, the detection method according to the present invention can be carried out more easily and simply. It is also preferable to kit various reagents, equipment, and the like used for the detection method according to the present invention in the probe set. In addition to the probe, the kit may include various buffers used for preparing the sample solution, an incubator attached with a thermostat used for denaturation treatment and associate formation, and the like.

### EXAMPLES

Next, the present invention will be described in more detail by showing examples and the like, but the present invention is not limited to the following examples.

### Example 1

Target nucleic acid molecules with an optional concentration were detected using two donor probes and one acceptor probe.

<1> Formation of Associate Between Two Donor Probes and One Acceptor Probe and Target DNA

Each of the following was dissolved in a reaction buffer (10 mM Tris-HCl, 400 mM NaCl, 0.05% Triton X-100) so as to obtain target DNA (5′-AGAGCTACGAGCTGCCTGACGGCCAGGTCATCACCATTGGCAATGAGCGG TTC-3′, SEQ ID NO: 1) at a final concentration of 0 to 100 mM; a donor probe a (5′-GAACCGCTCATITGCCAATGGTGATG-3′, SEQ ID NO: 2: the probe in which, in the second T, a fluorescent atomic group with two thiazole oranges is modified) at a final concentration of 20 nM; a donor probe b (5′-GTCAGGCAGCTCGTAGCTCTTCTCC-3′, SEQ ID NO: 3: the probe in which, in the second T, a fluorescent atomic group with two thiazole oranges is modified) at a final concentration of 20 nM; and an acceptor probe a (5′-ACCTGGCC-3′, SEQ ID NO: 4: the probe in which, in the fourth T, a fluorescent substance ATTO633 is modified, a base other than the fourth T is ENA) at a final concentration of 20 nM. Therefore a sample solution (sample solution 1-1) was prepared. The sample solution 1-1 thus prepared was incubated at 95° C. for 10 seconds using a thermal cycler. The temperature was lowered to 25° C., and the sample solution 1-1 was incubated for 30 minutes.

<2> Formation of Associate Between One Donor Probe and One Acceptor Probe and Target DNA

A sample solution (sample solution 1-2) was prepared in the same manner as in the <1> except that the donor probe b was not mixed thereinto, and an acceptor probe b (5′-ACCTGGCCGTCAGGCAGCTCGTAGCTCT-3′, SEQ ID NO: 5: the probe in which a fluorescent substance ATTO633 is modified at the 5′ terminal) was used instead of the acceptor probe a, as the acceptor probe. The sample solution 1-2 thus prepared was incubated at 95° C. for 10 seconds using a thermal cycler. The temperature was lowered to 25° C., and the sample solution 1-2 was incubated for 30 minutes.

<3> Measurement of Associate in Sample Solution by SSMC Associates containing target DNA in the sample solutions prepared in <1> and <2> above were measured by SSMC. Specifically, the associates of each sample solution were measured using a single-molecule fluorescence spectroscopy system MF-20 (Olympus Corporation) equipped with an optical system of a confocal fluorescence microscope and a photon-counting system, and therefore time-series light intensity data (photon count data) was obtained. The sample solution was excited with a laser with 488 nm and 300 μW, which is the excitation wavelength of thiazole orange, and fluorescence at 600 to 660 nm, which is the fluorescence wavelength of ATTO633, was detected using a bandpass filter. A light detection region in the sample solution was allowed to move at a moving rate of 15 mm/sec so as to perform measurement for 20 seconds. In addition, with BIN TIME set to 10μsec, peaks were detected by differentiation after smoothing the time-series light intensity data obtained by the measurement. Among the regions regarded as the peaks, peak intensities of the regions, which can approximate a Gaussian function and have intensity of 0.8 or more, were extracted.

The measurement results of the fluorescence luminance of each sample solution are shown in FIG. 2. In these results, in any of the sample solutions, it was observed that the fluorescence luminance of the fluorescence wavelength of ATTO633 tended to increase in a target DNA concentration-dependent manner. In particular, the fluorescent luminance was saturated when the concentration of the target DNA was 30 nM, and it was perceived that all the probes formed the associates with the target DNA. When comparing the fluorescence luminance of each of the sample solutions, in the sample solution 1-1 using the two donor probes at the saturation point (sample solution having the target DNA concentration of 30 nM), the fluorescence luminance was about 2 times the fluorescence luminance of the sample solution 1-2 using one donor probe. Therefore, fluorescence luminance was shown to increase by disposing two donor probes for one acceptor probe.

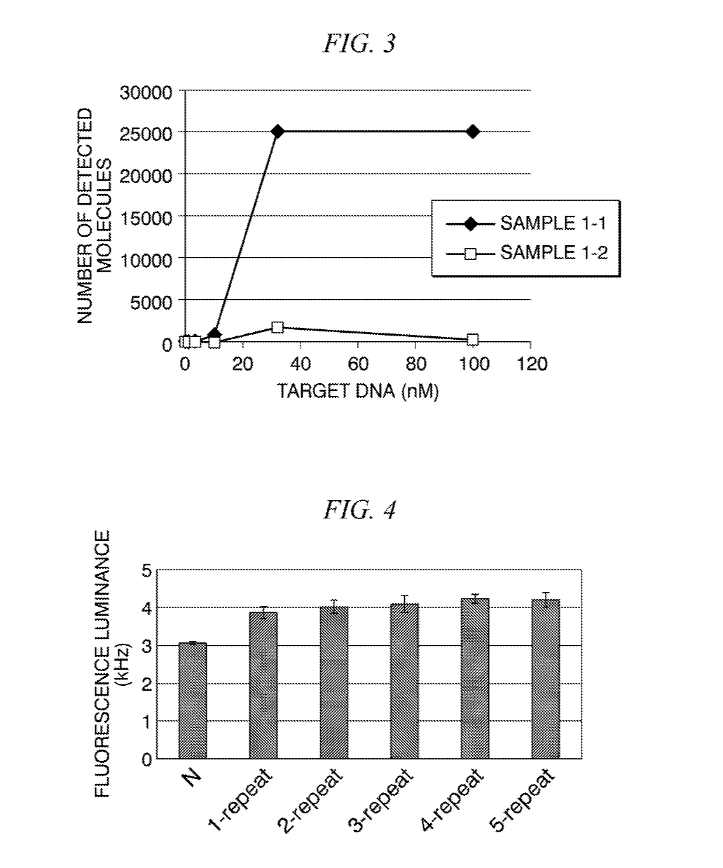

In addition, measurement results of the number of molecules of associates in each sample solution measured by SSMC are shown in FIG. 3. The number of detected molecules in SSMC in the sample solution 1-1 using the two donor probes at the saturation point (sample solution having the target DNA concentration of 30 nM) was about 10 times the number of detected molecules in the sample solution 1-2 using one donor probe. It was perceived that the reason is because the detection efficiency of one molecule was improved by an increase in the fluorescence intensity per molecule.

### Reference Example 1

The target nucleic acid molecule was detected by associating a plurality of donor probes tandemly on the same side of the acceptor probe.

As the target DNA, target DNA-1 (5′-AACTATACAACGGGCTGAA-3′, SEQ ID NO: 6), target DNA-2 (5′-AACTATACAACGGGCTGAAGGGCTGAA-3′, SEQ ID NO: 7), target DNA-3 (5′-AACTATACAACGGGCTGAAGGGCTGAAGGGCTGAA-3′, SEQ ID NO: 8), target DNA-4 (5′-AACTATACAACGGGCTGAAGGGCTGAAGGGCTGAAGGGCTGAA-3′, SEQ ID NO: 9), and target DNA-5 (5′-AACTATACAACGGGCTGAAGGGCTGAAGGGCTGAAGGGCTGAAGGGCTG AA-3′, SEQ ID NO: 10) were used. The target DNA-1 to target DNA-5 respectively associate so that 1 to 5 donor probes were in the same direction with respect to the acceptor probe.

Each of the following was dissolved in a reaction buffer (10 mM Tris-HCl, 400 mM NaCl, 0.05% Triton X-100) so as to obtain each target DNA at a final concentration of 10 mM, a donor probe (5′-TTCAGCCC-3′, SEQ ID NO: 11: the probe in which, in the fifth T, a fluorescent atomic group with two thiazole oranges is modified) at a final concentration of 20 nM; and an acceptor probe (5′-GTTGTATAGTT-3′, SEQ ID NO: 12: the probe in which a fluorescent substance ATTO633 is modified at the 5′ terminal) at a final concentration of 20 nM. Therefore a sample solution was prepared. Sample solutions containing the target DNA-1 to the target DNA-5 were used as sample solutions 2-1 to 2-5, respectively. As a control, a sample solution prepared in the same manner except that the target DNA was not added was used as a sample solution 2-0. Next, each sample solution was incubated at 95° C. for 10 seconds using a thermal cycler. The temperature was lowered to 25° C., and each sample solution was incubated for 30 minutes.

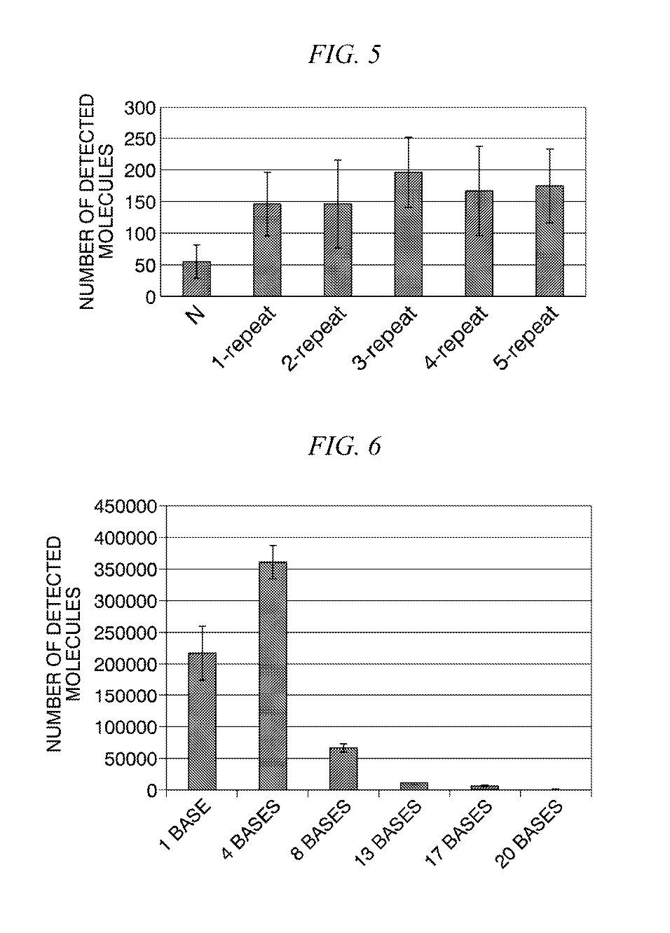

Thereafter, associates containing the target DNA in each sample solution were measured by SSMC in the same manner as in <3> of Example 1. FIG. 4 shows measurement results of the fluorescence luminance of each sample solution, and FIG. 5 shows measurement results of the number of molecules of the associate in each sample solution measured by SSMC. In FIGS. 4 and 5, “N” shows the result of the sample solution 2-0 and “l-repeat” to “5-repeat” show the results of the sample solution 2-1 to the sample solution 2-5, respectively. As a result, in the cases using any target DNA, the fluorescence luminance increased compared with the case of adding no target DNA (“N” in the drawing), but there was no difference in the fluorescence luminance in the sample solution 2-1 to the sample solution 2-5 (FIG. 4). Therefore, it was shown that even when a plurality of donor probes were disposed on one side of the acceptor probe, the fluorescence luminance released from the associate did not increase. In addition, as shown in FIG. 5, the same tendency was shown with respect to the number of detected molecules. In other words, it became clear that the effect of increasing the fluorescence luminance cannot be obtained unless the donor probes are disposed on both sides of the acceptor probe as in Example 1.

### Reference Example 2

The influence of a distance between the acceptor fluorescent substance and the donor fluorescent substance on the efficiency of FRET in the associate composed of the acceptor probe, the donor probe, and the target nucleic acid molecule was investigated.

<1> Formation of Associate in which Distance Between Acceptor Fluorescent Substance and Donor Fluorescent Substance is One Base on Target Nucleic Acid Molecule

Each of the following was dissolved in a reaction buffer (10 mM Tris-HCl, 400 mM NaCl, 0.05% Triton X-100) so as to obtain target DNA-1 (5′-TGAGOTAGTAGGTTGTATAGTT-3′, SEQ ID NO: 13) at a final concentration of 20 mM; a donor probe-1 (5′-CTACTACCTCA-3′, SEQ ID NO: 14: the probe in which, in the second T, a fluorescent atomic group with two thiazole oranges is modified) at a final concentration of 20 nM; and an acceptor probe-1 (5′-AACTATACAAC-3′, SEQ ID NO: 15: the probe in which a fluorescent substance ATTO633 is modified at the 3′ terminal) at a final concentration of 20 nM. Therefore a sample solution (sample solution 3-1) was prepared. Next, each sample solution was incubated at 95° C. for 10 seconds using a thermal cycler. The temperature was lowered to 25° C., and each sample solution was incubated for 30 minutes.

<2> Formation of Associate in which Distance Between Acceptor Fluorescent Substance and Donor Fluorescent Substance is Four Bases on Target Nucleic Acid Molecule

A sample solution (sample solution 3-2) was prepared in the same manner as in the <1> except that a donor probe-2 (5′-CTACTACCTCA-3′, SEQ ID NO: 14: the probe in which, in the fifth T, a fluorescent atomic group with two thiazole oranges is modified) was used instead of the donor probe-1, as a donor probe. Next, each sample solution was incubated at 95° C. for 10 seconds using a thermal cycler. The temperature was lowered to 25° C., and each sample solution was incubated for 30 minutes.

<3> Formation of Associate in which Distance Between Acceptor Fluorescent Substance and Donor Fluorescent Substance is Eight Bases on Target Nucleic Acid Molecule

A sample solution (sample solution 3-3) was prepared in the same manner as in the <1> except that a donor probe-3 (5′-CTACTACCTCA-3′, SEQ ID NO: 14: the probe in which, in the ninth T, a fluorescent atomic group with two thiazole oranges is modified) was used instead of the donor probe-1, as a donor probe. Next, each sample solution was incubated at 95° C. for 10 seconds using a thermal cycler. The temperature was lowered to 25° C., and each sample solution was incubated for 30 minutes.

<4> Formation of Associate in which Distance Between Acceptor Fluorescent Substance and Donor Fluorescent Substance is Thirteen Bases on Target Nucleic Acid Molecule

A sample solution (sample solution 3-4) was prepared in the same manner as in the <3> except that target DNA-2 (5′-GTTGTATAGTITGAGGTAGTAG-3′. SEQ ID NO: 16) was used instead of the target DNA-1, as the target nucleic acid molecule. Next, each sample solution was incubated at 95° C. for 10 seconds using a thermal cycler. The temperature was lowered to 25° C., and each sample solution was incubated for 30 minutes.

<5> Formation of Associate in which Distance Between Acceptor Fluorescent Substance and Donor Fluorescent Substance is Seventeen Bases on Target Nucleic Acid Molecule

A sample solution (sample solution 3-5) was prepared in the same manner as in the <2> except that target DNA-2 was used instead of the target DNA-1, as the target nucleic acid molecule. Next, each sample solution was incubated at 95° C. for 10 seconds using a thermal cycler. The temperature was lowered to 25° C., and each sample solution was incubated for 30 minutes.

<6> Formation of Associate in which Distance Between Acceptor Fluorescent Substance and Donor Fluorescent Substance is Twenty Bases on Target Nucleic Acid Molecule

A sample solution (sample solution 3-6) was prepared in the same manner as in the <1> except that target DNA-2 was used instead of the target DNA-1, as the target nucleic acid molecule. Next, each sample solution was incubated at 95° C. for 10 seconds using a thermal cycler. The temperature was lowered to 25° C., and each sample solution was incubated for 30 minutes.

<7> Measurement of Associate in Sample Solution by SSMC

Associates containing the target DNA in each sample solution were measured by SSMC in the same manner as in <3> of Example 1.

Measurement results of the number of molecules of associates in each sample solution measured by SSMC are shown in FIG. 6. In the drawing, “1 base,” “4 base,” “8 base,” “13 base,” “17 base,” and “20 base” indicate the results of the sample solutions 3-1 to 3-6, respectively. As shown in FIG. 6, a maximum number of detected molecules is shown in the sample solution 3-2. In the sample solution 3-3, the number of detected molecules which is 10% or more of the number of detected molecules of the sample solution 3-2 was maintained. However, the number of detected molecules was clearly small in the sample solutions 3-4 to 3-6. Based on these results, it was found that, in the target DNA, a distance (distance between pigments) between the base with which the base to which the donor fluorescent substance in the donor probe is bound is associated, and the base with which the base to which the acceptor fluorescent substance in the acceptor probe is bound is associated, affects detection efficiency of the target nucleic acid molecule; that the detection efficiency of the target nucleic acid molecule decreases as the distance between the pigments increases; that FRET is not perceived to be limited because a decrease in the number of detected molecules is small for a short distance between pigments; and that the distance between pigments is preferably 8 bases or less.

According to the method for detecting a target nucleic acid molecule according to the present invention, the target nucleic acid molecule present in the sample can be detected with high sensitivity and high accuracy, and thus can be utilized in the field of biochemistry, molecular biology, clinical examination, and the like, in which detection or quantitative analysis of nucleic acids in a sample is performed.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. A method for detecting a target nucleic acid molecule, comprising:
a step (a) of mixing, into a nucleic acid-containing sample, a first probe labeled with a first fluorescent substance which is an energy donor in a fluorescence resonance energy transfer phenomenon, a second probe labeled with a second fluorescent substance which is an energy acceptor in the fluorescence resonance energy transfer phenomenon, and a third probe labeled with the first fluorescent substance so as to prepare a sample solution;
a step (b) of allowing the target nucleic acid molecule in the sample solution prepared in the step (a) to associate with the first probe, the second probe, and the third probe so as to form an associate made of the first probe, the second probe, the third probe, and the target nucleic acid molecule; and
a step (c) of emitting light with an excitation wavelength of the first fluorescent substance to the sample solution after the step (b) so as to detect the target nucleic acid molecule using fluorescence released from the second fluorescent substance in the associate as an indicator,
wherein a region associating with the second probe is between a region associating with the first probe and a region associating with the third probe in the target nucleic acid molecule.

2. The method for detecting a target nucleic acid molecule according to claim 1,
wherein the first probe, the second probe, or the third probe is a probe in which emission luminance changes according to a state of associating or not associating with the target nucleic acid molecule.

3. The method for detecting a target nucleic acid molecule according to claim 2,
wherein the first fluorescent substance or the second fluorescent substance is a fluorescent atomic group exhibiting excitonic effects.

4. The method for detecting a target nucleic acid molecule according to claim 1,
wherein a distance between a base in the target nucleic acid molecule with which a base to which the first fluorescent substance in the first probe is bound is associated, and a base in the target nucleic acid molecule with which a base to which the second fluorescent substance in the second probe is bound is associated, is 8 bases or less, and
a distance between a base in the target nucleic acid molecule with which a base to which the first fluorescent substance in the third probe is bound is associated, and the base in the target nucleic acid molecule with which the base to which the second fluorescent substance in the second probe is bound is associated, is 8 bases or less.

5. The method for detecting a target nucleic acid molecule according to claim 1,
wherein a base length of the second probe is 5 to 17 bases.

6. The method for detecting a target nucleic acid molecule according to claim 1,
wherein a first target nucleic acid molecule that associates with the first probe, the second probe, and the third probe; and a second target nucleic acid molecule that binds to only one of the first probe and the third probe, and to the second probe are contained in the nucleic acid-containing sample,
in the step (b), a first associate obtained by associating the first target nucleic acid molecule with the first probe, the second probe, and the third probe; and a second associate obtained by associating the second target nucleic acid molecule with the second probe, and any one of the first probe and the third probe are formed, and
in the step (c), the light with the excitation wavelength of the first fluorescent substance is emitted to the sample solution, and the first target nucleic acid molecule and the second target nucleic acid molecule are distinctively detected using fluorescence luminance released from the second fluorescent substance in the associate of one molecule as an indicator so as to calculate an abundance ratio of the first target nucleic acid molecule and the second target nucleic acid molecule in the sample solution.

7. A probe set for detecting a target nucleic acid molecule, comprising:
a first probe in which a single-stranded nucleic acid molecule associating with the target nucleic acid molecule is labeled with a first fluorescent substance which is an energy donor in a fluorescence resonance energy transfer phenomenon,
a second probe in which a single-stranded nucleic acid molecule associating with the target nucleic acid molecule is labeled with a second fluorescent substance which is an energy acceptor in the fluorescence resonance energy transfer phenomenon; and
a third probe in which a single-stranded nucleic acid molecule associating with the target nucleic acid molecule is labeled with the first fluorescent substance,
wherein the first probe, the second probe, and the third probe are capable of forming, with the target nucleic acid molecule, an associate in which the third probe is disposed on a side opposite to the first probe based on the second probe, and
in a case where light with an excitation wavelength of the first fluorescent substance is emitted to the associate, a fluorescence resonance energy transfer occurs between the first fluorescent substance in the first probe and the second fluorescent substance in the second probe, and between the first fluorescent substance in the third probe and the second fluorescent substance in the second probe, and fluorescence released from the second fluorescent substance is detected.

8. The probe set according to claim 7,
wherein the first probe, the second probe, or the third probe is a probe in which emission luminance changes according to a state of associating or not associating with the target nucleic acid molecule.

9. The probe set according to claim 8,
wherein the first fluorescent substance or the second fluorescent substance is a fluorescent atomic group exhibiting excitonic effects.

10. The probe set according to claim 7,
wherein, in the associate,
a distance between a base in the target nucleic acid molecule with which a base to which the first fluorescent substance in the first probe is bound is associated, and a base in the target nucleic acid molecule with which a base to which the second fluorescent substance in the second probe is bound is associated, is 8 bases or less, and
a distance between a base in the target nucleic acid molecule with which a base to which the first fluorescent substance in the third probe is bound is associated, and the base in the target nucleic acid molecule with which the base to which the second fluorescent substance in the second probe is bound is associated, is 8 bases or less.

11. The probe set according to claim 7,
wherein a base length of the second probe is 5 to 17 bases.
